(19) United States
(12) Patent Application Publication
(10) Pub. No.: US 2016/0030600 A1
(43) Pub. Date: Feb. 4, 2016

(54) TARGETED DELIVERY OF NANOPARTICLES TO EPICARDIAL DERIVED CELLS (EPDC)

(71) Applicant: Crozet Medical GmbH, Duesseldorf (DE)

(72) Inventor: Juergen SCHRADER, Duesseldorf (DE)

(21) Appl. No.: 14/772,668

(22) PCT Filed: Mar. 6, 2014
(86) PCT No.: PCT/EP2014/000582

§ 371 Date: Sep. 3, 2015

Related U.S. Application Data
(60) Provisional application No. 61/773,880, filed on Mar. 7, 2013.

(51) Int. Cl. A61K 49/00; A61K 35/34; A61K 9/51; G01N 33/50; A61K 51/12

(52) U.S. Cl. CPC A61K 49/0093; G01N 33/5091; A61K 51/1244; A61K 9/51; A61K 35/34

(57) ABSTRACT

This invention relates to nanoparticles for use in the in vivo diagnostics of epicardial derived cells (EPDCs) to nano particles for use in the treatment of cardiac injury. The invention further relates to a method for analyzing EPDCs, to a method for labeling EPDCs, and to a method for transferring a therapeutic agent into an EPDC.

### FIELD OF THE INVENTION

This invention relates to nanoparticles for use in the in vivo diagnostics of epicardial derived cells (EPDCs) and to nanoparticles for use in the treatment of cardiac injury. The invention further relates to a method for analyzing EPDCs, to a method for labeling EPDCs, and to a method for transferring a therapeutic agent into an EPDC.

### BACKGROUND OF THE INVENTION

Acute myocardial infarction (MI) remains a leading cause of morbidity and mortality worldwide. Myocardial infarction occurs when myocardial ischemia, a diminished blood supply to the heart, exceeds a critical threshold and overwhelms myocardial cellular repair mechanisms designed to maintain normal operating function and homeostasis. Ischemia at this critical threshold level for an extended period results in irreversible myocardial cell damage or death.

Without immediate treatment, a myocardial infarction can cause permanent damage to substantial portions of the heart muscle, preventing efficient blood supply to the rest of the body and resulting in congestive heart failure. In addition, myocardial infarction can cause ventricular arrhythmias, in many cases resulting in cardiac arrest.

Thus, there exists a great need for novel therapies promoting repair of the injured heart tissue after myocardial infarction. Progress in developing new therapies hinges on understanding the myocardial injury response elicited by MI.

Only recently it was discovered that cardiac injury activates adult epicardial cells to respond by an epithelial-mesenchymal transition, forming epicardial derived cells (EPDCs). In response to injury, EPDCs reactivate the embryonic epicardial gene Wt1, expand in number and migrate into the underlying myocardium where they adopt a default fibroblast morphology or differentiate into vascular smooth muscle cells or cardiomyocytes. This raises the tantalizing possibility that EPDCs might be recruited for use in therapeutic myocardial regeneration. It is, however, unknown, which factors determine the fate of epicardial derived cells. Approaches for efficiently reprogramming epicardial derived cells into cardiomyocytes to promote cardiac regeneration are unknown in the art.

Improved understanding of the pathophysiology of the MI response will be fundamental for the development of novel regenerative therapy approaches. A major obstacle to investigating the MI response has been an inability to specifically trace EPDCs after myocardial infarction in vivo, which could provide unique insights into the differentiation and migration of these stem cells, elucidating their biological role in the course of myocardial injury.

Zhou et al. (J Clin Invest. 2011; 121(5): 1894-1904) describes irreversible labeling of adult epicardial cells and their derivatives using Cre-IoxP-based approaches in a mouse model.

EP 2 152 369 B1 relates to the labeling of circulating monocytes using fluorine-containing compounds for diagnostically detecting inflammatory processes.

### OBJECTS OF THE INVENTION

It was an object of the invention to provide means for imaging epicardial derived cells. Furthermore, the present invention aims to provide novel approaches for the treatment of cardiac injury. Such methods and compositions for use in such diagnostic and therapeutic applications would offer major advantages for improving the treatment options for MI patients.

### SUMMARY OF THE INVENTION

Surprisingly, it has been found that epicardial derived cells, which are newly formed after myocardial infarction, are highly phagocytic and avidly take up nanoparticles after intravenous injection. The phagocytic potential of EPDCs, which was first discovered by the inventors of the present invention, was surprisingly found to allow for targeted delivery of active agents such as labeling agents and therapeutic agents to EPDCs. Thus, it was surprisingly found that the phagocytic potential of EPDCs can be exploited for imaging epicardial derived cells and for regenerative treatment of cardiac injury.

Thus, in one aspect, the present invention relates to a nanoparticle comprising one or more labeling agent(s) for use in the in vivo diagnostics of EPDCs.

In particular embodiments of the present invention, the in vivo diagnostics is/are in vivo imaging.

In particular embodiments, said one or more labeling agent(s) is/are independently selected from a fluorine-containing compound, a fluorescent compound, and a genetic label.

In particular embodiments, said fluorine-containing compound is selected from organic and inorganic perfluorinated compounds.

In particular embodiments, said organic perfluorinated compound is a perfluorocarbon, particularly a perfluorocarbon selected from perfluorooctyl bromide, perfluorooctane, perfluorodecalin and perfluoro-15-crown-5-ether, more particularly, said organic perfluorinated compound is perfluorooctyl bromide.

In particular embodiments, said in vivo diagnostics are performed by means of magnetic resonance imaging, in particular 19F magnetic resonance imaging.

In particular embodiments, said fluorine-containing compound comprises at least on ¹⁸F isotope.

In particular embodiments, said in vivo diagnostics are performed by PET scanning, in particular by 18F PET scanning.

In another aspect, the present invention relates to a nanoparticle comprising one or more therapeutic agent(s) for use in the treatment of a cardiac disorder, particularly cardiac injury, cardiac ischemia or myocardial infarction.

In particular embodiments, said treatment comprises the differentiation of EPDCs into cardiomyocytes and/or vascular smooth muscle cells.

In particular embodiments, said one or more therapeutic agent(s) is/are one or more cardiomyocyte differentiation factor(s) and/or one or more vascular smooth muscle cell differentiation factor(s).

In particular embodiments, said one or more cardiomyocyte differentiation factor(s) and/or one or more vascular smooth muscle cell differentiation factor(s) is/are independently selected from a peptide, a protein, a nucleic acid encoding a peptide, a protein or a nucleic acid with specificity for a target nucleic acid, a nucleic acid with specificity for a target nucleic acid, and a small molecule.

In particular embodiments, said protein is selected from a transcription factor, a growth factor, a cytokine, a chemokine, and thymosin β4.

In particular embodiments, said nucleic acid encoding a peptide, a protein or nucleic acid with specificity for a target nucleic acid, is selected from a nucleic acid encoding a transcription factor, a growth factor, a cytokine, a chemokine, thymosin β4, and a miRNA.

In particular embodiments, said transcription factor is selected from GATA4, HAND2, MEF2C, Tbx5, Myocd, and BAF60C.

In particular embodiments, said growth factor is selected from transforming growth factors, particularly from TGF-β and BMP.

In particular embodiments, said the nucleic acid encoding a peptide, a protein or nucleic acid with specificity for a target nucleic acid is operatively linked with an EPDC-specific promoter, particularly an EPDC-specific promoter selected from the WT-1 promoter, the Tbx18 promoter, the Raldh-1 promoter, the Raldh-2 promoter, and the PDGF-α promoter.

In particular embodiments, said nucleic acid with specificity for a target nucleic acid is selected from a miRNA, and an siRNA.

In particular embodiments, said miRNA is selected from miRNAs 1, 132, 133, 208, 212, and 499.

In particular embodiments, said small molecule is selected from vitamins and ascorbic acid and retinoic acid inhibitors, particularly BMS 189453.

In particular embodiments, said nanoparticle has a size from about 100 nm to about 400 nm.

In particular embodiments, said nanoparticle is selected from a lipid-based and a polymer-based nanoparticle, in particular, said nanoparticle is selected from liposomes, polymer-drug conjugates, polymeric nanoparticles, micelles, dendrimers, polymerosomes, protein-based nanoparticles, biological nanoparticles such as viral and bacterial nanoparticles, inorganic nanoparticles and hybrid nanoparticles.

In particular embodiments, said nanoparticle is an unilamellar or a multilamellar liposome.

In particular embodiments, said one or more labeling agent(s) or said one or more therapeutic agent(s) is/are formulated as from about 0.5% to about 50%, particularly from about 1% to about 30%, more particularly form about 5% to about 20% of said labeling agent(s) or said therapeutic agent(s) emulsified in a lipid solution comprising lecithin, particularly purified egg lecithin.

In particular embodiments, said nanoparticle further comprises an EPDC targeting moiety.

In particular embodiments, said EPDC targeting moiety is a surface structure allowing for targeting of EPDCs via epitopes of antigens, receptors or other proteins, and non-proteinaceous membrane compounds of said EPDCs.

In particular embodiments, said nanoparticle is for intravenous administration, injection into the pericardial sac via a catheter or injection into the injured myocardium via a catheter, particularly for intravenous administration.

In particular embodiments, said nanoparticle is administered after from about one to about five days after cardiac injury, particularly from about 2 to about 4 days after cardiac injury, most particularly after from about 3 to about 4 days after cardiac injury.

In another aspect, the present invention relates to a method for analyzing EPDCs comprising the step of detecting the presence or absence of a label in EPDCs contacted with a nanoparticle according to the present invention in vitro.

In particular embodiments, the method of the present invention further comprises the step of contacting EPDCs with a nanoparticle according to the present invention in vitro.

In another aspect, the present invention relates to a method for labeling EPDCs comprising the step of contacting EPDCs in vitro with a nanoparticle according to the present invention.

In another aspect, the present invention relates to a method for in vivo imaging of EPDCs by 19F magnetic resonance imaging or 18F PET scanning comprising the step of administering a nanoparticle according to the present invention by intravenous injection.

In another aspect, the present invention relates to a method for transferring one or more therapeutic agent(s) into an EPDC comprising the step of contacting said EPDC in vitro with a nanoparticle according to the present invention.

In another aspect, the present invention relates to an EPDC comprising one or more therapeutic agent(s).

In another aspect, the present invention relates to a pharmaceutical composition comprising the EPDC cell of the present invention.

In another aspect, the present invention relates to the EPDC of the present invention or the pharmaceutical composition of the present invention for use as a medicament.

In another aspect, the present invention relates to a method for diagnosing EPDCs comprising the step of administering a nanoparticle according to the present invention to a patient.

In another aspect, the present invention relates to a method for treating a cardiac disorder/injury comprising the step of administering a nanoparticle according to the present invention to a patient.

### BRIEF DESCRIPTION OF THE FIGURES

FIG. 1 shows pulse labeling of EPDCs in rats after myocardial infarction with PFC containing nanoparticles. The PFC emulsion was intravenously injected (2 ml, 10% PFC emulsion into the rats 3 days after myocardial infarction. (a) Representative ¹⁹F-MR images at day 7 (4 days post MI) revealed labeling predominately of the epicardial layer in several heart section (S5-S9, see FIG. 4). ¹⁹F-labeling extends beyond the infarcted area as measured by sirius red staining for collagen. (b) When PFC containing nanoparticles were tagged with rhodamine (rho-PFC), the fluorescence pattern was similar, showing both epicardial and intramyocardial distribution of rho-PFC. Electron microscopy of an epicardial cell shows substantial cellular uptake of PFC containing nanoparticles (130 nm) as well as coated vesicles (CV) and collagen fibers (CF). Vesicles appear empty due to the washout of PFC during the fixation process (some vesicles are marked by asterisks). (c) time course (n=3) of free plasma PFC nanoparticles when intravenously injected=(2 ml, 15% PFC emulsion). Inset shows representative ¹⁹F-MR images of plasma samples collected at different time points.

FIG. 2 shows the dynamics of epicardial labeling with rhodamine tagged PFC emulsion (Rho-PFC). (a) Rho-PFC was injected on day 3 after myocardial infarction (MI) and heart samples were analyzed after 12 hours (D4), 4 days (D7) and 10 days (D14), respectively. Fluorescence microscopy analysis revealed that the fluorescence within the epicardial layer decreased over time, while fluorescence intensity within the infarcted myocardium proportionally increased (b and c), although the epicardial layer maintained its thickness over the period analyzed (d). Interestingly, rho-PFC was found on day 7 (4 days post PFC injection) to form lumen-like structures resembling small vessels within. (b). Histological analyses showed that rhodamine-positive vessels within the infarcted area constituted about 10% of total vessels stained positive for smooth muscle actin ((e) and FIG. 8).

FIG. 3 shows that EPDCs exhibit a stem cell-like expression pattern and phagocytotic activity. Immunohistochemical staining of heart sections (a) and ex vivo (b) immune staining of EPDCs harvested 7 days after myocardial infarction shows a distinct expression of different nuclear and cytoplasmic antigens typical for progenitor cells (WT-1, Flk-1) indicating proliferation (Ki-67) and future destination (sm-actin, PDGFR-α) (n=3-5; white bars equals 10 mm). (d) EPDCs devoid of CD45⁺ cells and CD11b⁺ cells from the infarcted heart were incubated with rhodamine tagged PFC nanoparticles for up to 120 min. Uptake was determined at 37° C. by washing with ice-cold PBS and mean fluorescence intensity was measured by FACS. Data are the mean±SD, analyzed applying one-way ANOVA with repeated measurements and Dunnett's post test. *** P<0.0001.

FIG. 4 shows the quantification of ¹⁹F distribution in the outer, mid and inner wall of the left ventricle from apex to base. Four days after intravenous administration of the PFC emulsion (day 7 after MI; conditions as in FIG. 1) hearts were briefly perfused with saline medium and then fixed with 4% PFA. Analysis of the ¹⁹F signal in heart sections from the apex to the base demonstrate significantly higher ¹⁹F signal in the outer proportion of the left ventricle (S5-S10; mainly epicardial layer) in comparison to the mid and inner part.

FIG. 5 shows the labeling of the epicardial layer in the mouse heart after MI. The mouse hart was subjected to 60 min ischemia (LAD) followed by reperfusion. Nanoemulsion (500 μl PFC) was given intravenously on day 4 and the ex vivo ¹⁹F image analysis was performed on day 7. The labeling pattern of the epicardial layer after MI was similar to that in rats (see FIG. 1).

FIG. 6 shows electron microscopy of the epicardial layer and immune cells within the infarcted myocardium. (a) epicardial cell fully loaded with nanoparticles. (b) two cells with an elongated nucleus resembling smooth muscle cells. (c) immune cell (*) within the epicardial layer migrating out of the lumen of a venule (d) elongated/corkscrew shape of the nucleus of a smooth muscle cell containing nanoparticles (→). Mast cells (*) (e). Immune cell within the injured myocardium loaded with nanoparticles. (f) PFC-loaded immune cells within the injured myocardium adjacent to a plasma cell (*).

FIG. 7 shows the preferential labeling of Immune cells by administering PFCs briefly after MI. PFCs were administered as early as 24 hours after MI and ¹⁹F-MRI was performed 4 days later. When PFCs were applied, the epicardial cells only started to proliferate and therefore remained unlabeled due to the short plasma half life of emulsified PFCs. PFC-Iabeled monocytes, however, remain in the circulation for about 3 days and migrate into the injured myocardium for the days to follow. S5-S8 refers to the section number from apex to base similar to experiments reported in FIG. 4.

FIG. 8 shows the phenotypic analysis of rhodamine labeled cells within the injured myocardium. Cryosections of the heart were stained with antibodies against smooth muscle actin (sm-actin) and cardiac troponin T. (a) Rho-PFC positive cells were found to form a lumen structure and co-stained with sm-actin. (b) rhodamine stains the entire sm-actin positive vessel including a side branch. (c) very rarely, some Rho-PFC positive cells within the infarcted area were found to also stain for cardiac troponin T.

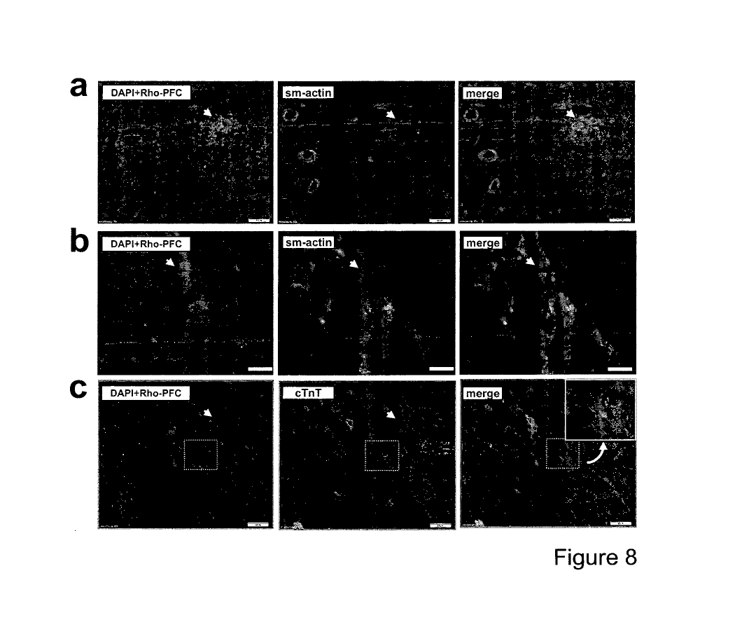

FIG. 9 shows the results of in vivo 19F MRI of a mouse injected with PFC containing nanoparticles after myocardial infarction. The thorax cross section shows the circular heart muscle, lung tissue, lung vessels, aorta, the spinal cord, bones and muscle tissue. A strong 19F signal could be observed in the infarcted region, resulting from phagocytic uptake of PFC containing nanoparticles by EPDCs and, presumably, also phagocytic monocytes. Areas devoid of phagocytic cells showed no 19F signal.

### DETAILED DESCRIPTION OF THE INVENTION

The present invention may be understood more readily by reference to the following detailed description of the invention and the examples included therein.

In one aspect, the present invention relates to a nanoparticle comprising one or more labeling agent(s) for use in the in vivo diagnostics of EPDCs.

In another aspect, the present invention relates to a nanoparticle comprising one or more therapeutic agent(s) for use in the in treatment of a cardiac disorder, particularly cardiac injury, cardiac ischemia or myocardial infarction.

Both uses are based on the fact that epicardial derived cells (EPDCs) are highly phagocytic, which was first discovered by the inventors of the present invention. It was found that administration, for example by intravenous injection, of phagocytable particles, such as nanoparticles selected from, inter alia, liposomes and polymer-based nanoparticles, results in targeted delivery of these phagocytable particles to EPDCs. Targeted delivery of phagocytable particles to EPDCs can be exploited in the delivery of therapeutic agents to EPDCs, for instance in regenerative therapy approaches for cardiac injury and in the delivery of labeling agents to EPDCs, which is for instance applicable in in vivo imaging of these stem cells. Selective labeling of adult epicardial cells and their derivatives has so far only been achieved by irreversible genetic modification in a knock in mouse model using the Cre-loxP technology (Zhou et al., J Clin Invest. 2011; 121(5): 1894-1904). The present invention provides novel means for the targeted delivery of any active agent, including inter alia labeling agents and therapeutic agents, to native EPDCs, either in vivo or in vitro.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps or components, or groups thereof. The term “comprising” thus includes the more restrictive terms “consisting of” and “consisting essentially of”.

In the context of the present invention, the term “nanoparticle” refers to biocompatible delivery vehicles with a diameter up to about 600 nm, e.g. with a diameter from about 100 nm to about 400 nm, preferably with a diameter from about 100 nm to about 400 nm. The nanoparticles are phagocytable by epicardial derived cells. Suitable nanoparticles are neither limited to any specific composition nor any specific morphology. Suitable nanoparticles may be of any physicochemical structure, comprising but not being limited to liposomes, polymer-drug conjugates, polymeric nanoparticles, micelles, dendrimers, polymerosomes, protein-based nanoparticles, biological nanoparticles such as viral and bacterial nanoparticles, inorganic nanoparticles and hybrid nanoparticles. Nanoparticles may be lipid-based (e.g., liposomes and micelles) or polymer-based (e.g., polymer-drug conjugates, polymeric nanoparticles, dendrimers, polymerosomes, protein-based nanoparticles, and also micelles). Nanoparticles may be neutral, cationic or anionic, depending on their composition. Nucleic acids are preferably delivered via cationic nanoparticles, e.g., cationic liposomes (i.e. lipoplexes) or cationic polymer-based nanoparticles (i.e. polyplexes). They may, however be delivered via anionic or neutral nanoparticles using cationic bridging agents (e.g., calcium or cationic poly-L-lysine).

The nanoparticle surface may be functionalized by various methods to modulate drug release, residence time in the blood, distribution, and targeting of tissues or specific cell surface antigens with a targeting ligand. In particular, nanoparticles may comprise surface structures allowing for targeting of epicardial derived cells. Fusogenic lipids such as DOPE may be incorporated into liposomes in order to enhance endosomal escape. Nanoparticle surfaces may be decorated by polymers such as poly-ethylene glycol (PEG) in order to prolong circulation in the blood by reducing liposome recognition and uptake by reticulo-endothelial cells. Further, nanoparticle surfaces, may be functionalized by peptides that are “markers for self”, e.g., CD47 peptides, in order to decrease macrophage-mediated clearance of nanoparticles.

In the context of the present invention, the term “about” or “approximately” means within 20%, alternatively within 10%, including within 5% of a given value or range.

In the context of the present invention, the term “epicardial derived cell” or “EPDC” refers to an adult or somatic multipotent stem cell, or progenitor cell, which originates from an adult epicardial cell that has undergone an epithelial-to-mesenchymal transition or EMT, typically as a response to cardiac injury. EPDCs have the capacity to self-renew and to differentiate into diverse specialized cell types, namely fibroblasts, vascular smooth muscle cells and cardiomyocytes.

As used herein, the term “differentiation” refers to the adaptation of cells to a specific function. Differentiation leads to a more committed cell, meaning that the cell loses its potency, i.e. its ability to differentiate into different cell types.

The epicardial derived cell to be targeted by the nanoparticle of the present invention may be of any origin, the epicardial derived cell is particularly mammalian, more particularly human.

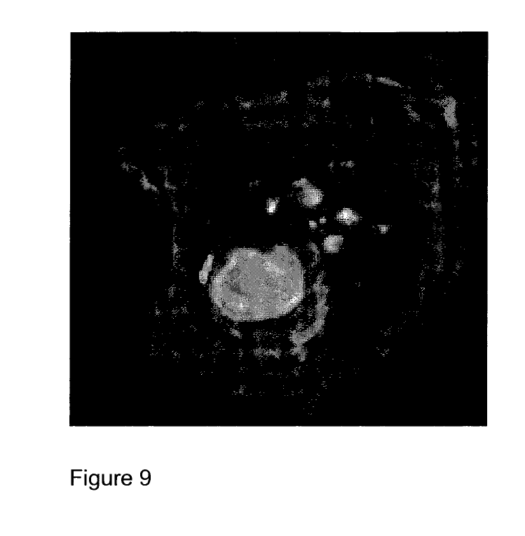

In the context of the present invention, the term “targeted delivery” means that the nanoparticles are preferentially taken up, particularly in a phagocytic process, by epicardial derived cells compared to non-phagocytic reference cells. In the context of the present invention, the term “targeted delivery” or “preferential uptake” is defined as more than about 50 times more efficient uptake of nanoparticles by epicardial derived cells than by non-phagocytic reference cells, particularly more than about 100 times, more particularly more than about 500 times more efficient uptake of nanoparticles by epicardial derived cells than by non-phagocytic reference cells as assessed by in vitro uptake studies. In vitro uptake studies are well known in the art. In vitro uptake studies may be performed according to the following protocol: suspended cells are exposed to 1% FITC-coupled PFC emulsions in MACS buffer for various time periods ranging from 5 to 120 min at 37° C. The termination of uptake is achieved by washing three times with ice-cold PBS for 5 min at 500 g. In order to assess nanoparticle uptake, mean fluorescence intensity (MFI) is measured in the FITC channel in a FACS Canto II flow cytometer (BD Bioscience). Nanoparticle uptake is assessed by Fluorescence Measurement, e.g. by using FACS. Targeted nanoparticle delivery to phagocytic cells, in particular EPDCs and, presumably, also phagocytic monocytes has been demonstrated by in vivo magnetic resonance tomography in mice (see FIG. 9). Areas devoid of phagocytic cells showed no 19F signal.

In the context of the present invention, the term “targeted delivery” or “preferential uptake” is further defined as comparable uptake of nanoparticles by epicardial derived cells and phagocytic CD11b⁺ cells. In this context, “comparable uptake” is defined to mean that the uptake of nanoparticles by epicardial derived cells is from about 0.25 to about 6 times the uptake of nanoparticles by phagocytic CD11 b⁺ cells, particularly from about 0.1 to about 20 times, more particularly from about 0.5 to about 10 times, most particularly from about 0.5 to about 4 times the uptake of nanoparticles by phagocytic CD11 b⁺ cells, as assessed by in vitro uptake studies.

The nanoparticles of the present invention may also be for use in the targeted delivery to other phagocytic cells, e.g., cells that have undergone an EMT (epithelial-to-mesenchymal transition) such as cancer cells, and somatic or adult stem cells, which are activated upon tissue injury and represent sources used to rebuild damaged tissues. Such adult stem cells are for example present in kidney and articular cartilage. Undergoing an epithelial-to-mesenchymal transition may render these cells phagocytic and thus targetable by the nanoparticles of the present invention. The nanoparticles of the present invention may be for use in in vivo diagnostics of cells that have undergone an EMT. The nanoparticle of the present invention may also be for use in the treatment of tissue injury, in particular, they may be for use in the targeted delivery of one or more therapeutic agents, e.g., differentiation factors, to adult stem cells activated after tissue injury. Further, the nanoparticles of the present invention may also be for use in cancer therapy, in particular they may be for use in the targeted delivery of cancer therapeutics to cancer cells.

In particular embodiments of the present invention, the in vivo diagnostics is/are in vivo imaging.

In particular embodiments, said one or more labeling agent(s) is/are independently selected from a fluorine-containing compound, a fluorescent compound, and a genetic label.

In the context of the present invention, the term “genetic label” refers to a nucleic acid sequence encoding a gene product, preferably a protein, which can be used to label a cell. Genetic labels include but are not limited to nucleic acid sequences encoding fluorescent proteins and antigens, in particular transposons carrying nucleic acid sequences encoding fluorescent proteins or antigen epitopes, most particularly transposons carrying nucleic acid sequences encoding GFP (transposon-GFP).

The nucleic acid sequence that is incorporated into the nanoparticles may be derived from any species and does not necessarily have to be a wild-type sequence as long as the gene product can function as a label, i.e. is fluorescent in the case of fluorescent proteins or can be bound by a specific antibody in the case of antigens. The nucleic acid sequence may harbor nucleotide exchanges, insertions or deletions. The nucleic acid sequence may further comprise a sequence encoding a tag or a signaling peptide mediating the translocation of the gene product to the plasma membrane.

The nucleic acid sequence is preferably operatively linked with a promoter sequence that allows transcription mediated by a DNA dependent RNA polymerase in the target EPDC. The promoter sequence is selected for efficient transcription of the DNA in the target EPDC. The promoter sequence may be a heterologous promoter sequence for the given target EPDC, e.g. the viral CMV promoter or the viral S40 promoter. Alternatively, the promoter sequence may be a EPDC specific promoter, such as the WT-1 promoter, the Tbx18 promoter, the Raldh-1 promoter, the Raldh-2 promoter, and the PDGF-α promoter. The nucleic acid sequence preferably further comprises a polyadenylation signal at the 3′ end.

Fluorine-containing compounds allow for the use of devices available and familiar in the clinic, namely the use of MR spectrometers for magnetic resonance imaging.

In particular embodiments, said fluorine-containing compound is selected from organic and inorganic perfluorinated compounds.

In particular embodiments, said organic perfluorinated compound is a perfluorocarbon, particularly a perfluorocarbon selected from perfluorooctyl bromide, perfluorooctane, perfluorodecalin and perfluoro-15-crown-5-ether, most particularly perfluorooctyl bromide.

In the context of the present invention, the term “perfluorocarbons” comprises organofluorine compounds that contain only carbon and fluorine, such as perfluorooctane or perfluorodecalin, as well as fluorocarbon derivatives, such as Perflouorooctyl bromide and perfluoro-15-crown-5-ether.

In particular embodiments, said in vivo diagnostics are performed by means of magnetic resonance imaging.

In particular embodiments, said fluorine-containing compound comprises at least on ¹⁸F isotope. The presence of at least one ¹⁸F isotope allows for the use of devices available and familiar in the clinic, namely the use of PET Scanners for PET-scanning.

In particular embodiments, said in vivo diagnostics are performed by PET scanning.

Imaging may be performed in vivo or in vitro, for example on heart preparations. Imaging may be performed for research purposes. For example, imaging of epicardial derived cells may allow for tracking of these cells after myocardial infarction in order to study their migration, differentiation and biological role in the course of myocardial injury.

In another aspect, the present invention relates to a nanoparticle comprising one or more therapeutic agent(s) for use in the treatment of a cardiac disorder, particularly cardiac injury, cardiac ischemia or myocardial infarction.

In particular embodiments, said treatment comprises the differentiation of EPDCs into cardiomyocytes and/or vascular smooth muscle cells.

In particular embodiments, said one or more therapeutic agent(s) is/are one or more cardiomyocyte differentiation factor(s) and/or one or more vascular smooth muscle cell differentiation factor(s).

In the context of the present invention, the term “cardiomyocyte differentiation factor” is intended to refer to any agent, which can convert an epicardial derived cell into a cardiomyocyte, either by itself or in combination with other agents.

In the context of the present invention, the term “vascular smooth muscle cell differentiation factor” is intended to refer to any agent, which can convert an epicardial derived cell into a smooth muscle cell of an artery, either by itself or in combination with other agents.

In particular embodiments, said one or more cardiomyocyte differentiation factor(s) and/or one or more vascular smooth muscle cell differentiation factor(s) is/are independently selected from a peptide, a protein, a nucleic acid encoding a peptide, a protein or a nucleic acid with specificity for a target nucleic acid, a nucleic acid with specificity for a target nucleic acid, and a small molecule.

In particular embodiments, said protein is selected from a transcription factor, a growth factor, a cytokine, a chemokine, and thymosin β4.

In particular embodiments, said nucleic acid encoding a peptide, a protein or nucleic acid with specificity for a target nucleic acid, is selected from a nucleic acid encoding a transcription factor, a growth factor, a cytokine, a chemokine, thymosin β4, and a miRNA.

In particular embodiments, said transcription factor is selected from GATA4, HAND2, MEF2C, Tbx5, Myocd, and BAF60C.

In particular embodiments, said growth factor is selected from transforming growth factors, particularly TGF-β and BMP.

The nucleic acid sequence is preferably operatively linked with a promoter sequence that allows transcription mediated by a DNA dependent RNA polymerase in the target EPDC. The promoter sequence is selected for efficient transcription of the DNA in the target EPDC. The promoter sequence may be a heterologous promoter sequence for the given target EPDC, e.g. the viral CMV promoter or the viral S40 promoter.

In particular embodiments, said the nucleic acid encoding a peptide, a protein or nucleic acid with specificity for a target nucleic acid is operatively linked with an EPDC-specific promoter, particularly an EPDC-specific promoter selected from the WT-1 promoter, the Tbx18 promoter, the Raldh-1 promoter, the Raldh-2 promoter, and the PDGF-α promoter.

The nucleic acid sequence preferably further comprises a polyadenylation signal at the 3′ end.

The nucleic acid sequence that is incorporated into the nanoparticle, for example the nucleic acid encoding a peptide, a protein or nucleic acid with specificity for a target nucleic acid or the nucleic acid with specificity for a target nucleic acid such as a miRNA, may be derived from any species, particularly from human or other mammals, depending on the application and the target EPDC. The nucleic acid sequence does not necessarily have to be a wild-type sequence as long as the nucleic acid sequence itself in the case of a nucleic acid with specificity for a target nucleic acid or its gene product in the case of a nucleic acid encoding a peptide, a protein or nucleic acid with specificity for a target nucleic acid shows functional activity comparable to the wild-type nucleic acid sequence or gene product. The nucleic acid sequence may harbor nucleotide exchanges, insertions or deletions. The nucleic acid sequence may further comprise a sequence encoding a tag, for instance a VP16 tag.

In particular embodiments, said nucleic acid with specificity for a target nucleic acid is selected from a miRNA, and an siRNA.

In particular embodiments, said miRNA is selected from miRNAs 1, 132, 133, 208, 212, and 499.

In particular embodiments, said small molecule is selected from vitamins and ascorbic acid and retinoic acid inhibitors, particularly BMS 189453

In one embodiment, the nanoparticle comprises two or more different cardiomyocyte differentiation factors and/or vascular smooth muscle cell differentiation factors. Alternatively, two or more different nanoparticles each comprising at least one cardiomyocyte differentiation factor and/or at least one vascular smooth muscle cell differentiation factor can be used concurrently. Transcription factor encoding genes can thus be delivered in combination with small molecules or miRNAs, in order to increase the efficiency of cardiomyocyte differentiation.

In particular embodiments, said nanoparticle has a size from about 100 nm to about 400 nm. In the context of the present invention, the term “size from about 100 nm to about 400 nm” means “about 100 nm to about 400 nm in diameter”.

In particular embodiments, said nanoparticle is selected from a lipid-based and a polymer-based nanoparticle, in particular, said nanoparticle is selected from liposomes, polymer-drug conjugates, polymeric nanoparticles, micelles, dendrimers, polymerosomes, protein-based nanoparticles, biological nanoparticles such as viral and bacterial nanoparticles, inorganic nanoparticles and hybrid nanoparticles.

In particular embodiments, said nanoparticle is an unilamellar or a multilamellar. Liposomes and their generation are well known in the art (Mozafari M R, Liposomes: an overview of manufacturing techniques. Cell Mol Biol Lett 10 (2005) 711-9; Basu S C, Basu M, Methods in Molecular Biology: Liposomes Methods and Protocols, Humana Press Inc., Totowa, N. J., 2002).

Preferably, the liposomes have a size which is suitable for the cellular uptake by epicardial derived cells. Typically the size of suitable liposomes is from about 50 nm or 75 nm or 100 nm or 150 nm or 200 nm to about 600 nm or 500 nm or 400 nm or 350 nm or 300 nm or 250 nm, particularly from about 100 nm to about 400 nm.

In particular embodiments, said one or more labeling agent(s) or said one or more therapeutic agent(s) is/are formulated as from about 0.5% to about 50%, particularly from about 1% to about 30%, more particularly form about 5% to about 20% of said labeling agent(s) or said therapeutic agent(s) emulsified in a lipid solution comprising lecithin, particularly purified egg lecithin.

In particular embodiments, said nanoparticle further comprises an EPDC targeting moiety.

In particular embodiments, said EPDC targeting moiety is a surface structure allowing for targeting of EPDCs via epitopes of antigens, receptors or other proteins, and non-proteinaceous membrane compounds of said EPDCs.

In particular embodiments, said targeting moieties comprise but are not limited to peptides, nucleic acids, antibodies or antibody fragments, carbohydrates or small molecules and specifically bind to epitopes of antigens, receptors or other proteins, and non-proteinaceous membrane compounds of said EPDCs.

Said peptides and nucleic acids may be aptamers, i.e. molecules that bind to a specific target molecule via their 3D configuration. Their target molecules comprise inter alia proteins and amino acids. Dissociation constants of aptamers typically lie within the picomolar to nanomolar range. Aptamers thus bind to their target molecules comparably strong as antibodies. Aptamers are usually created by selecting them in vitro from a large random sequence pool, but natural aptamers also exist.

In the context of the present invention, the term “antibody” refers to an immunoglobulin (Ig) molecule that is defined as a protein belonging to the class IgG, IgM, IgE, IgA, or IgD (or any subclass thereof), which includes all conventionally known antibodies and functional fragments thereof. The antibody may be a monoclonal antibody, a polyclonal antibody, a recombinantly produced antibody, including a recombinantly produced chimeric or humanized antibody, or a fully synthetic antibody. A “functional fragment” of an antibody/immunoglobulin molecule hereby is defined as a fragment of an antibody/immunoglobulin molecule (e.g., a variable region of an IgG) that retains the antigen-binding region. An “antigen binding region” of an antibody typically is found in one or more hypervariable region(s) (or complementarity-determining region, “CDR”) or an antibody molecule, i.e. the CDR-1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320). A preferred class of antibody molecules for use in the present invention is IgG.

“Functional fragments” include the domain of a F(ab′)2 fragment, a Fab fragment, scFv or constructs comprising single immunoglobulin variable domains or single domain antibody polypeptides, e.g. single heavy chain variable domains or single light chain variable domains. The F(ab′)2 or Fab may be engineered to minimize or completely remove the intermolecular disulphide interactions that occur between the CH1 and CL domains.

An antibody may be derived from immunizing an animal, or from a recombinant antibody library, including an antibody library that is based on amino acid sequences that have been designed in silico and encoded by nucleic acids that are synthetically created. In silico design of an antibody sequence is achieved, for example, by analyzing a database of human sequences and devising a polypeptide sequence utilizing the data obtained therefrom. Methods for designing and obtaining in silico created sequences are described, for example, in Knappik et al, J. Mol. Biol. (2000) 296:57; Krebs et al., J. Immunol. Methods. (2001) 254:67; and U.S. Pat. No. 6,300,064 issued to Knappik et al.

In the context of the present invention, a molecule is “specific for”, “specifically recognizes” or “specifically binds to” a target molecule, such as epitopes of antigens, receptors or other proteins, and non-proteinaceous membrane compounds of said EPDCs, if such a molecule is able to discriminate between such a target molecule and one or more reference molecule(s), since binding specificity is not an absolute, but a relative property. In its most general form (and when no defined reference is mentioned), “specific binding” refers to the ability of the molecule to discriminate between the target molecule of interest and an unrelated biomolecule, as determined, for example, in accordance with a specificity assay as known in the art. Such methods comprise, but are not limited to Western blots, ELISA, RIA, ECL, IRMA tests and peptide scans. For example, a standard ELISA assay can be carried out. The scoring may be carried out by standard color development (e.g. secondary antibody with horseradish peroxide and tetramethyl benzidine with hydrogen peroxide). The reaction in certain wells is scored by the optical density, for example, at 450 nm. Typical background, i.e. the negative reaction, may be about 0.1 OD; typical positive reaction may be about 1 OD. This means that the ratio between a positive and a negative score can be 10-fold or higher. Typically, determination of binding specificity is performed by using not a single reference biomolecule, but a set of about three to five unrelated biomolecules, such as milk powder, BSA, transferrin or the like.

In particular embodiments, said nanoparticle is for intravenous administration, injection into the pericardial sac via a catheter or injection into the injured myocardium via a catheter, particularly for intravenous administration.

The nanoparticle of the present invention can be provided as solution, suspension, lyophilisate or any alternative form. It can be provided in combination with agents for the adjustment of the pH value, buffers, agents for the adjustment of toxicity, and such.

The appropriate nanoparticle dose depends on the application (i.e. in vivo or in vitro methods of EPDC labeling or differentiating EPDCs into cardiomyocytes), species, physical condition and weight of the subject, the form of administration and the composite. The administration can be carried out once or several times, dependent on the application.

The nanoparticle of the present invention is suitable for applications in human and veterinary medicine. In particular, it can be used for regenerative treatment of cardiac injury.

In particular embodiments, said nanoparticle is administered after from about one to about five days after cardiac injury, particularly from about two to about 4 days after cardiac injury, most particularly after from about 3 to about 4 days after cardiac injury.

The inventors of the present invention have found that labeling of epicardial cells by nanoparticle administration was dependent on the time point of nanoparticle administration. Nanoparticle administration at day 3 or 4 after MI in a rat model resulted in efficient pulse labeling of epicardial derived cells. At this time point, epicardial derived cells have already proliferated for about 2 to 3 days and the epicardial layer has already grown to a thickness of 120 μm. In contrast, nanoparticle administration 24 hours after MI when epicardial cells only start to proliferate, does not label the epicardial cell layer but preferentially labels immune cells.

In another aspect, the present invention relates to a method for analyzing EPDCs comprising the step of detecting the presence or absence of a label in EPDCs contacted with a nanoparticle according to the present invention in vitro.

In particular embodiments, the method of the present invention further comprises the step of contacting EPDCs with a nanoparticle according to the present invention in vitro.

In another aspect, the present invention relates to a method for labeling EPDCs comprising the step of contacting EPDCs in vitro with a nanoparticle according to the present invention.

In another aspect, the present invention relates to a method for in vivo imaging of EPDCs by 19F magnetic resonance imaging or 18F PET scanning comprising the step of administering a nanoparticle according to the present invention by intravenous injection.

In another aspect, the present invention relates to a method for transferring one or more therapeutic agent(s) into an EPDC comprising the step of contacting said EPDC in vitro with a nanoparticle according to the present invention.

In a particular embodiment, said one or more therapeutic agent(s) is/are (a) cardiomyocyte differentiation factor and/or (a) vascular smooth muscle cell differentiation factor(s) and the method according to the present invention comprises the differentiation of said EPDC into a cardiomyocyte or a vascular smooth muscle cell.

In a particular embodiment, the in vitro method comprising the differentiation of said EPDC into a cardiomyocyte further comprises the steps of providing an EPDC from a donor, and culturing said EPDC, after contacting it with a nanoparticle according to the present invention, under conditions effective to allow differentiation of said EPDC into a cardiomyocyte and/or to allow the cell to expand.

In another aspect, the present invention relates to an EPDC comprising one or more therapeutic agent(s). In a particular embodiment, said EPDC is prepared by the method of the present invention.

The cells obtained by the method according to the present invention may for example be used in regenerative medicine for the treatment of cardiac injury.

In another aspect, the present invention relates to a pharmaceutical composition comprising the EPDC cell of the present invention.

The pharmaceutical composition can be in the form of a solution, a suspension or any other suitable form. Typically, the composition further comprises a pharmaceutically acceptable carrier, diluent, and/or excipient. Agents for adjusting the pH value, buffers, agents for adjusting toxicity, and the like may also be included. The composition can be administered by the usual routes. Preferably, a therapeutically effective dose is administered to the subject, and this dose depends on the particular application, the subject's weight and state of health, the manner of administration and the formulation, etc. Administration can be single or multiple, as required.

In the context of the present invention, the term “pharmaceutically acceptable” refers to molecular entities and other ingredients of pharmaceutical compositions that are physiologically tolerable and do not typically produce untoward reactions when administered to a mammal (e.g., human). The term “pharmaceutically acceptable” may also mean approved by a regulatory agency of a Federal or a state government or listed in the U.S. Pharmacopeia or other generally recognized pharmacopeia for use in mammals, and more particularly in humans.

The pharmaceutical composition is suitable for applications in human and veterinary medicine. In particular, it can be used for regenerative treatment of cardiac injury.

In another aspect, the present invention relates to the EPDC of the present invention or the pharmaceutical composition of the present invention for use as a medicament.

In another aspect, the present invention relates to a method for diagnosing EPDCs comprising the step of administering a nanoparticle according to the present invention to a patient

In another aspect, the present invention relates to a method for treating a cardiac disorder/injury comprising the step of administering a nanoparticle according to the present invention to a patient.

The invention is now described with reference to the following examples: These examples are provided for the purpose of illustration only and the invention should not be construed as being limited to these examples, but rather should be construed to encompass any and all variations which become evident as a result of the teaching provided herein.

### EXAMPLES

### Example 1

### Pulse Labeling of EPDCs after Myocardial Infarction with PFC Containing Nanoparticles

Induction of cardiac ischemia and reperfusion was performed in accordance with the national guidelines on animal care (Guide of the care and use of laboratory animals, 8^{th} Edition, National Research Council of the National Academies) as previously described. Male rats (Wistar, 200-250 g body weight, 12-16 weeks of age) used in this study were bred at the Tierversuchsanlage of Heinrich-Heine-Universität, Düsseldorf, Germany and fed with a standard chow diet and received tap water ad libitum. Male Wistar rats (250-320 g) were intubated and anaesthetized by mechanical ventilation with isoflurane (1.5% v/v; Abbott, Wiesbaden, Germany) in 100% oxygen at a rate of 80 strokes/min and a tidal volume of 3 ml. Animals were placed in a supine position with paws taped to an electrocardiogram (ECG) board (lead II) to measure S-T segment elevations during the induction of myocardial infarction. The chest was then opened with a lateral cut along the left side of the sternum. The pericardium was then gently dissected to allow visualization of coronary artery anatomy. Ligation was preceded with a 6-0 polypropylene suture with a tapered needle passed underneath the LAD, 2-3 mm from the tip of the left auricle. The success of occlusion of the LAD was verified visually under the microscope by the absence of blood flow in the epicardium as well significant elevations of S-T segment. The occlusion was maintained for as long as 60 min until the suture was released. Thereafter, the chest was closed with one layer through the muscle and a second layer through the skin.

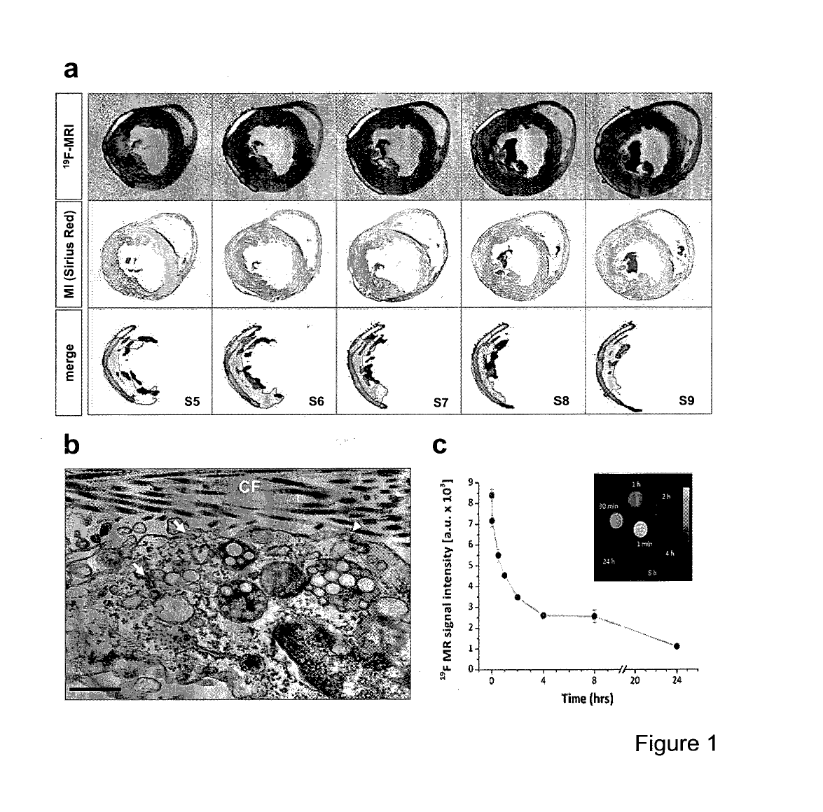

For tagging immune and epicardial cells, a bolus injection of total volume of 2 ml emulsified perfluorocarbons (10% PFCs) was given intravenously 3 days after ischemia under a temporary anesthesia with isoflurane (2.0% v/v) using a homemade mouse mask. To analyze the PFC uptake pharmacokinetics after injection, blood samples were taken immediately (1 min) and at various time points up to 24 hours. After separation from blood cells, free PFC in the 200 μl plasma samples were measured by ¹⁹F MRI and a time course of free plasma PFC nanoparticles was assessed (See FIG. 1c). Analysis of the PFC plasma concentration after intravenous injection revealed an exponential decrease, the half-life being about 2 hours. This strongly suggests that resident epicardial cells were pulse-labeled by the fee plasma PFCs. To detect PFC-tagged cells in the heart, rats were euthanized 3 days after PFC injection and the hearts was fixed with 4% fresh paraformaldehyde in 0.1 M PBS for 2 hours before ¹⁹F MRI measurement.

MRI measurements were performed on a Bruker AVANVEIII 9.4 Tesla Wide Bore (89 mm) NMR spectrometer operating at frequencies of 400.13 MHz for 1H and 376.46 MHz for 19F measurements using Paravision 5.1 as operating software. A Bruker microimaging unit (Mini 2.5) equipped with an actively shielded 57-mm gradient set (capable of 1 T/m maximum gradient strength and 110 μs rise time at 100% gradient switching) was used. The fixed hearts were placed in a home-build adapter and inserted into a 25-mm resonator tuneable to 1H and 19F. After acquisition of morphological 1H images, the resonator was tuned to 19F and anatomically matching 19F images were recorded using a 3D RARE sequence (RARE factor 32, FOV 25.6×25.6×20 mm3, matrix 64×64×20, resulting in a voxel size after zero filling of 0.2×0.2 mm2 in-plane, slice thickness 1 mm, TR 2.5 s, TE 4.78 ms, 8 averages, acquisition time, 13.20 min). For merging of 1H and 19F data, the hot iron color look-up table of Paravison was applied to 19F MR images.

Isotropic high resolution 3D data sets were acquired from a FOV of 20×20×20 mm3 using matrices of 256×256×256 for both 1H and 19F. For further processing reconstructed 1H and 19F image stacks were imported into the 3D visualization software Amira (Mercury Computer Systems). 1H signals were associated to the respective anatomical structures using the Segmentation Editor of Amira. For segmented areas, individual surfaces were calculated with unconstrained smoothing. Subsequently, surface views with a semi-transparent display using a “fancy” were created. For overlay, anatomic corresponding 19F data were volume rendered by the Voltex Module of Amira. The default colormap (red) and rgba lookup mode were used for visualization, and the resulting projection from the “shining” data volume was computed using an intensity range of 5000-30000. Fade-in of the projection and concomitant rotation of the surface views were coordinated with the DemoMaker of Amira.

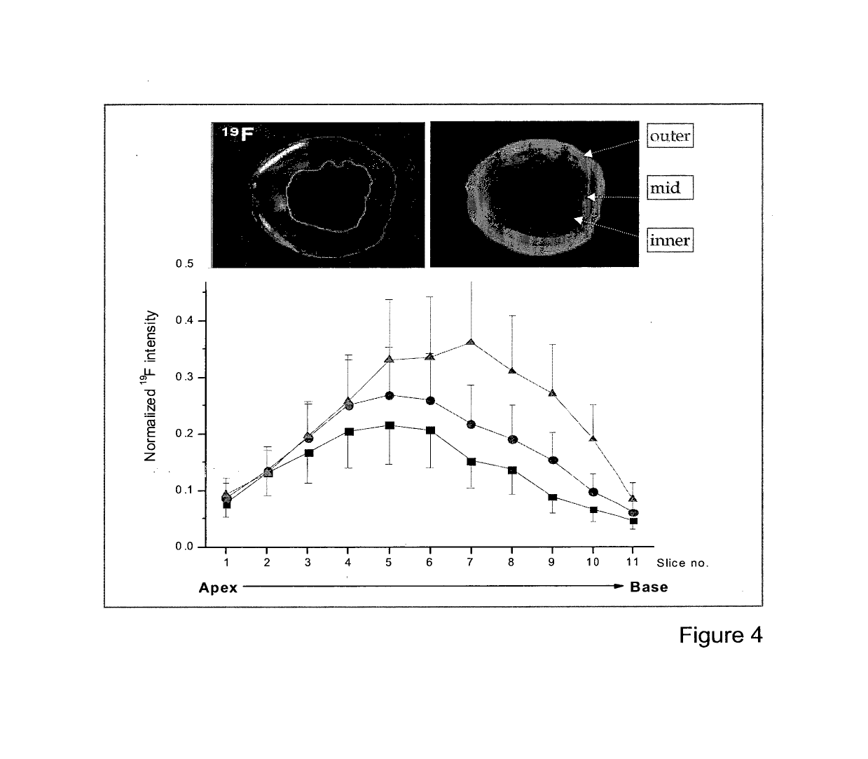

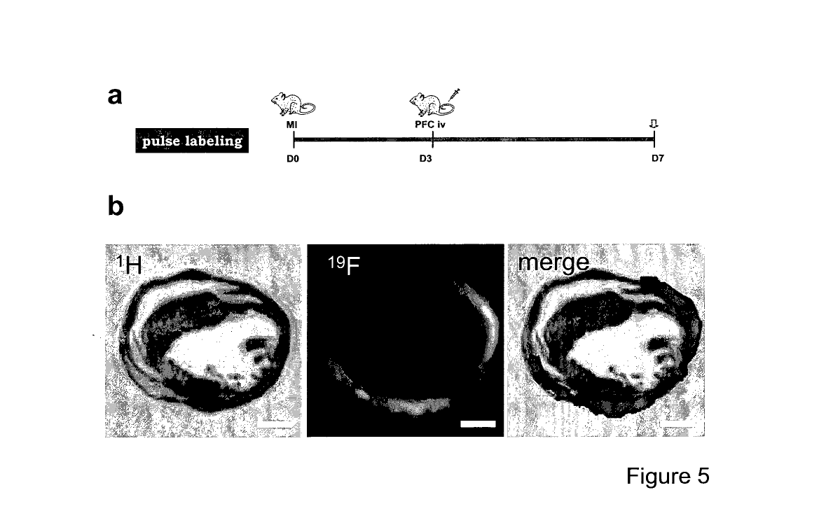

Representative ¹⁹F-MR images at day 7 (4 days post MI) revealed labeling predominately of the epicardial layer in several heart sections. ¹⁹F-labeling extended beyond the infarcted heart area as measured by Sirius red staining for collagen (See FIG. 1a) and spanned several slices of the infarcted area. The epicardial ¹⁹F-signal was significantly stronger than the middle and inner layer of the infarcted heart when sectioning the heart in 11 slices from apex to base (See FIG. 4). A similar epicardial labeling pattern was also observed after ischemia/reperfusion in mice (see FIG. 5) and is therefore not species specific.

### Example 2

### Pulse Labeling of EPDCs after Myocardial Infarction with Rhodamine-Labeled PFC Containing Nanoparticles

Alternatively, rhodamine-labeled. PFCs (2 ml) were given intravenously 3 days after ischemic injury in order to verify the localization of PFC-tagged cells in the heart by fluorescence microscopy. Induction of cardiac ischemia/reperfusion was performed as described above (Example 1). To detect rhodamine-labeled PFC containing cells in the heart, rats were euthanized 3 days after rhodamine-PFC injection and the hearts were cryopreserved. Cryopreserved heart samples were cut into 8 μm slices. To avoid a dissociation of rhodamine label and markers of the initial PFC carrier due to downstream processes after infiltration, all slides were air dried and red fluorescence images were immediately recorded without further processing because of water solubility of rhodamine-labeled PFCs. For further immunostaining, the tissue slices were fixed for 10 min in Zamboni's fixative and rinsed thrice with PBS and then blocked in 5% BSA in 0.05 M TBS for 1 hour at room temperature. The primary antibodies including the anti-mouse-smooth muscle actin antibody (sm-actin, 1:400) and anti-cardiac troponin T (cTnT, 1:400) in 0.8% BSA in TBS were incubated with tissue samples overnight at 4° C. After three washing steps with PBS containing 0.1% saponin, the secondary antibodies goat anti-rabbit-Ig and goat anti-mouse-Ig (1:400, Dako, Hamburg, Germany) were used in 0.8% BSA for staining of sections while nuclei were counterstained with 4,6-diamidino-2-phenylindole (DAPI, Sigma). Data were acquired with fluorescent microscopy equipped with standard filter sets (MX 61, Olympus) and analyzed with a software of AnalySIS (Olympus).

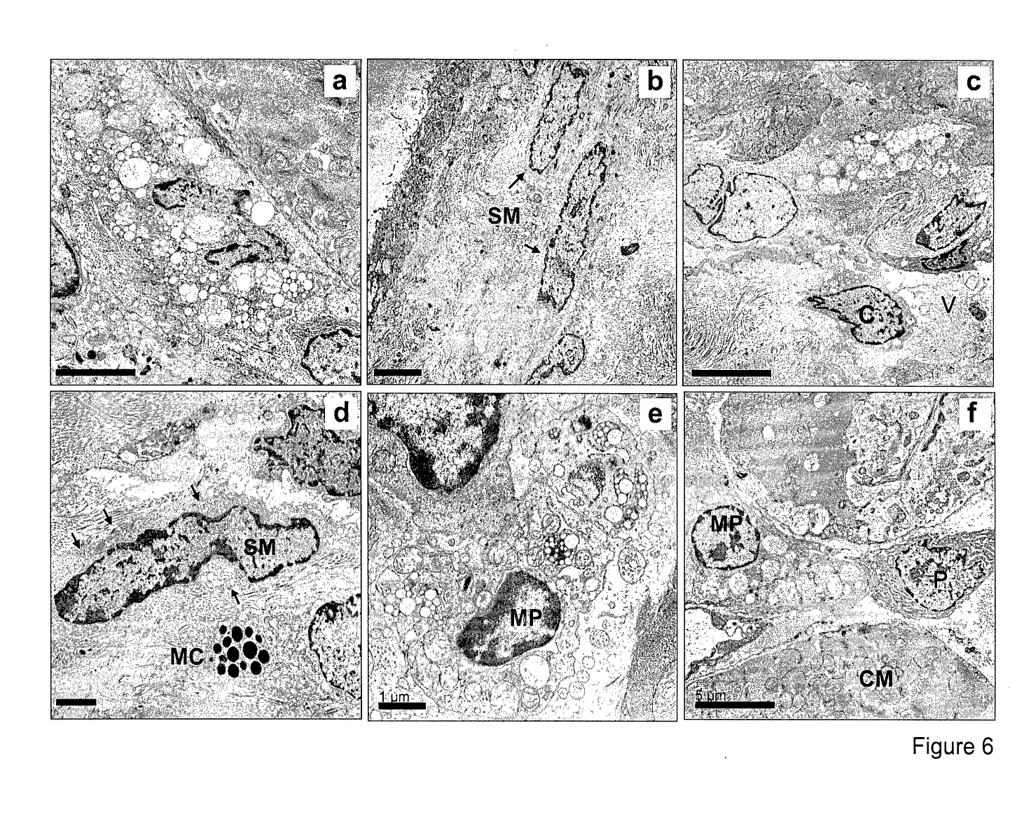

The fluorescence pattern was similar to the ¹⁹F-MR pattern showing both epicardial and intramyocardial distribution of rhodamine-labeled PFCs. Electron microscopy of epicardial cells revealed substantial cellular uptake of PFC containing nanoparticles which in part were clustered into multilaminar endosomes (See FIG. 1b and FIG. 6). Endocytic epicardial cells showed coated vesicles and the size of the PFC containing liposomes found by EM was similar to the diameter of the PFC emulsion (130 nm). Vesicles appeared empty due to the washout of PFC during the fixation process. The epicardial layer also contained′ cells with structural features of smooth muscle cells such as elongated or corkscrew shape of the nucleus (see FIG. 6b, 6d). EM revealed venules at the epicardium/myocardial interface with occasionally immune cells migrating out of the vessel lumen (see FIG. 6c) and mast cells within the epicardium (see FIG. 6d). As expected, liposomes associated with immune cells within the infarcted area were also found (see FIG. 6e, 60.

### Example 3

### Time Point of PFC Injection—Optimization

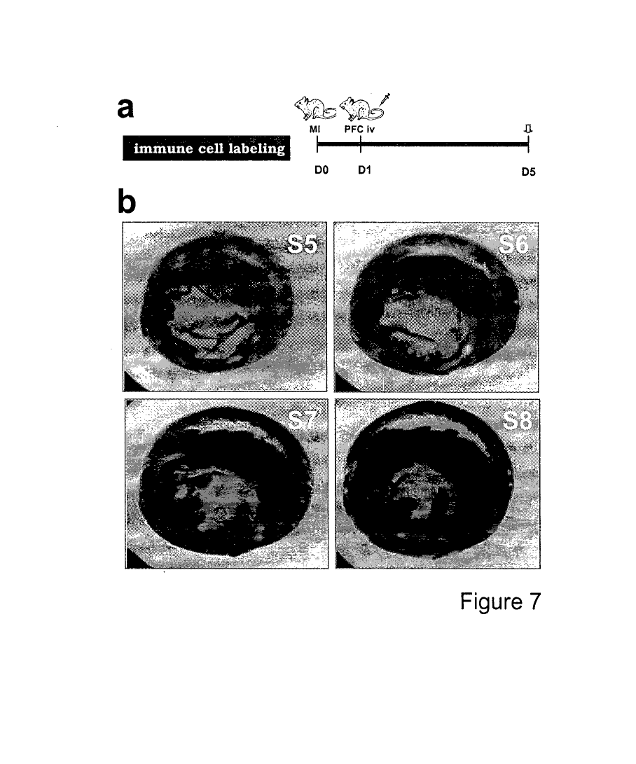

Induction of cardiac ischemia/reperfusion was performed as described above (Example 1). 2 ml emulsified perfluorocarbons (10% PFCs) were given intravenously 24 hours after ischemic injury and ¹⁹F-MRI was performed 4 days later as described above (Example 1). 24 hours after ischemic injury, the epicardial cells only start to proliferate. ¹⁹F-MRI revealed that the epicardial cells remained unlabeled, presumably due to the short plasma half-life of emulsified PFCs. In contrast, administering PFCs shortly after MI preferentially labeled immune cells. PFC-labeled monocytes remained in the circulation for about 3 days and migrated into the injured myocardium for the days to follow. At day 5 after MI, the ¹⁹F-MRI signal integrated the accumulation of labeled macrophages over time. Under these conditions, the ¹⁹F signal was associated mainly with the mid- and endomyocardial layer, the site of injury and monocyte accumulation (see FIG. 7).

### Example 4

### Dynamics of Epicardial Labeling with Rhodamine Tagged PFC Emulsion (Rho-PFC)

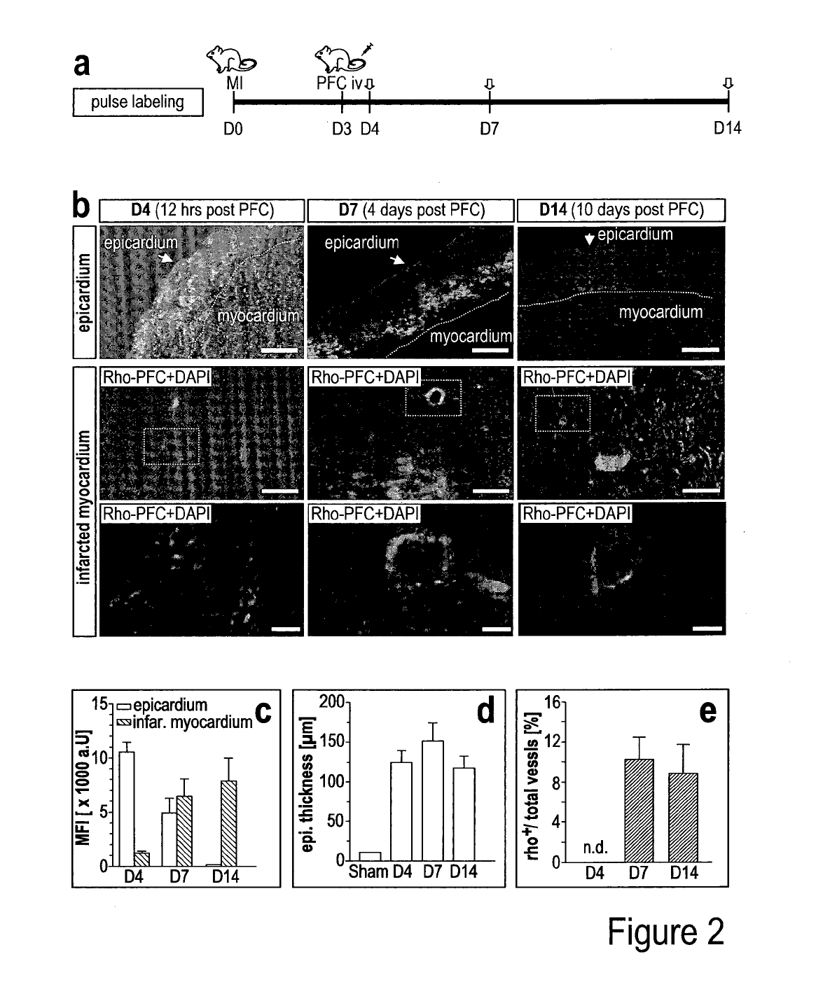

Induction of cardiac ischemia/reperfusion was performed as described above (Example 1). Rhodamine-labeled PFCs (2 ml) were given intravenously 3 days after ischemic injury. Heart samples were collected at early stage (12 hours after Rho-PFC injection—day 3 after MI), later stage (4 days after Rho-PFC injection—day 6 after MI) and long-term stage (10 days after Rho-PFC injection—day 14 after MI). Immunofluorescence Microscopy was performed as described above (Example 2). When hearts were harvested 12 hours after injections of nanoparticles, corresponding to day 4 after MI, the majority of the fluorescent label was associated with the epicardial cell layer which stained the entire layer in a somewhat patchy fashion (see FIG. 2a, b). Three days later, corresponding to day 7 after MI, the outer side of the epicardial layer had lost part of its fluorescent label and mean fluorescence intensity within the injured heart was increased (see FIG. 2b,d). Interestingly, at this point of time, smooth muscle cells of large vessels surrounding the infarcted area clearly showed rhodamine fluorescence which comprised about 10% of all large vessels within this area (see FIG. 2c). At day 14 after MI (day 10 after PFC application) the epicardial layer was almost fully devoid of fluorescence, while fluorescently labeled large vessels are still clearly visible (FIG. 2b, c). These experiments demonstrate that tracking of epicardial cells after being labeled with nanoparticles is possible.

### Example 5

### Characterization of EPDCs

Immunohistochemistry was performed on the epicardial layer 7 days after MI. Immunohistochemistry identified cells positive for WT1 and PDGFR-α, two established markers of epicardial derived cells. Furthermore, KI-67, a nuclear protein that is associated with cellular proliferation, mainly stained cells in the outer part of the epicardial cell layer, suggesting that EPDC may be primarily formed in this region prior to their migration into the injured heart.

To further characterize individual cells labeled with PFCs ex vivo, EPDCs devoid of CD45⁺ cells were isolated by means of a newly established procedure. 12 hours prior to tissue digestion animals were injected with PFCs as described above. After rapid excision of the heart from the thorax, the heart was perfused according to Langendorff for 3 minutes (perfusion pressure 80-100 mmHg, 37° C.) with an oxygenated medium containing 4.0 mM NaHCO3, 10.0 mM HEPES, 30.0 mM 2,3-butanedion-monoxime, 11.0 mM glucose, 0.3 mM EGTA, 126.0 mM NaCl, 4.4 mM KCl and 1.0 mM MgCl2×6 H₂O to free it from blood. Then tissue digestion of the epicardial layer of the heart was performed by bathing the heart in medium containing 1200 IU/ml collagenase II (BioChrom AG, Berlin, Germany) under continuous rotation with 12 rpm at 37° C. for 20 minutes. Digestion procedure was stopped by the addition of 3 ml FCS. The heart was discarded and the resulting cell suspension was meshed through a 40 μm cell strainer (BD Falcon). After centrifugation at 700 g for 7 minutes supernatant was discarded and pellet was resuspended in MACS buffer for further staining.

Cells were incubated with FcR-blocking reagent (mouse anti-rat CD32, BD Bioscience) at 4° C. for 5 minutes and stained for CD45 (APC-Cy7, mouse anti-rat, BD Bioscience, 1:100) thereafter. After 10 minutes of incubation at room temperature cells were washed with MACS buffer, centrifuged at 700 g for 7 minutes and resuspended in 80 μl MACS buffer for MACS depletion. MACS microbeads depletion was performed according to manufacturer's protocols. Briefly, 20 μl anti-PE microbeads (MACS miltenyi Biotec) were added to the cell suspension and incubated at 4° C. for 15 minutes. Hereafter, cells were washed, centrifuged (700 g for 7 minutes), supernatant was discarded and cells were resuspended in 500 μl MACS buffer. Then cells were loaded to the depletion column (MS column, MACS miltenyi Biotec) and collected after depletion of leukocytes labeled for CD45-PE. Quantification of effective leukocyte depletion was performed using a FACS Canto II flow cytometer (BD Bioscience).

The resulting cell suspension devoid of leukocytes was loaded to a custom-made cytospin apparatus to enrich and adhere cells on a poly-L-lysine coated slide (Polysine, ThermoScientific). In brief, 100-200 μl were loaded to the cytospin machine and centrifuged at 320 g for 5 minutes. Supernatant was discarded, and the resulting glass slides air-dried and fixed with 4% PFA for the next step of immunostaining.

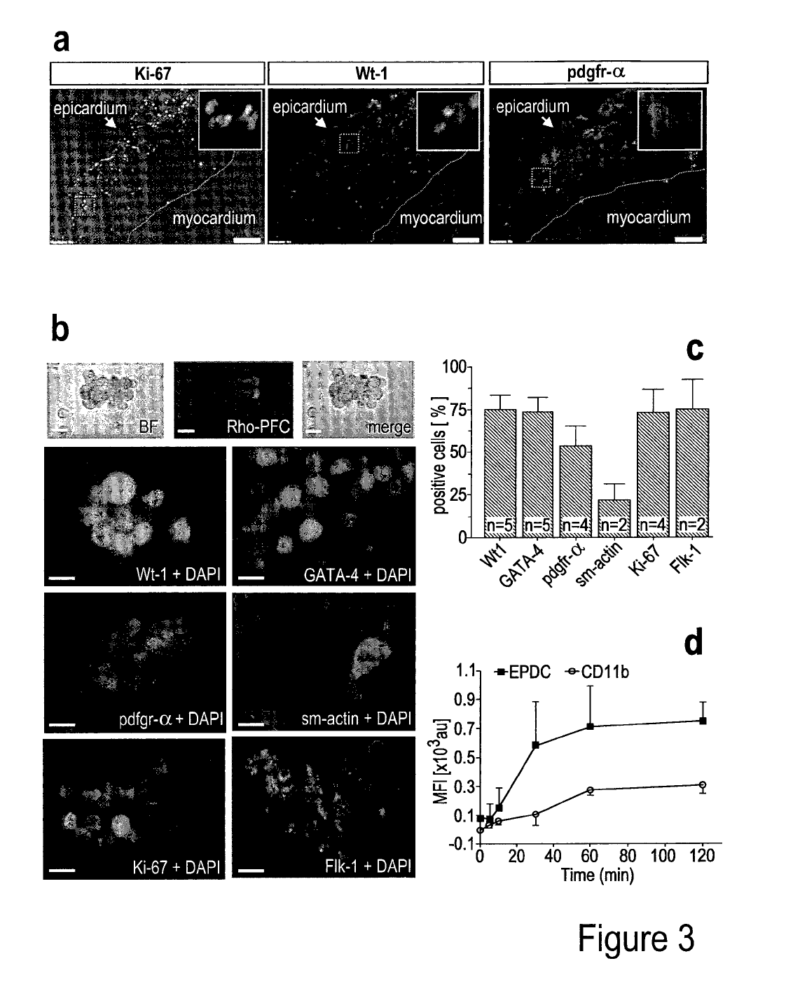

Cells were stained for anti-Wilms tumor-1 (WT-1, 1:100), anti-GATA-4 (GATA-4, 1:100), anti-platelet derived growth factor receptor alpha (PDGFR-α, 1:100), anti-smooth muscle actin (sm-actin, 1:100), anti-Ki-67 (Ki-67, 1:100) and anti-FIk-1 (Flk-1, 1:100) as described above (Example 2) except for using 0.1% Triton-100 instead of saponin for nuclear permeabilization in WT-1 staining. Data were acquired with a fluorescent microscope (MX 61, Olympus) and recorded using a digital camera (UC30, Olympus). Per antibody per animal cells were counted in five fields of view with a 20fold magnification. About 75% of the analyzed cells were positive for WT-1, PDGFR-α, Ki-67 and Flk-1, while about 50% of the cells stained for PDGFR-α. Interestingly, about 28% of the epicardial cells were positive for smooth muscle actin (see FIG. 3b).

To verify that the cell isolation procedure allowed for recovery of epicardial cells previous labeled in vivo with rhodamine containing PFCs, fluorescence associated with the epicardium-derived cell suspension was measured as described above (Example 2). Analysis revealed that in two experiments (Rhodamine-labeled PFCs applied on day 3 after MI and epicardium digested on day 7) about 90% of all cells recovered after cytospin were positive for rhodamine fluorescence. This convincingly demonstrates that the in vivo signal—either assessed by ¹⁹F-MRI or fluorescence—was derived predominantly from cells exhibiting all characteristic markers of EPDCs.

### Example 6

### Ex Vivo Uptake of PFCs

To demonstrate that the freshly isolated epicardial cells also have the ability to phagocytize PFCs, uptake studies were performed and the kinetics were compared with isolated CD11b⁺ cells. Uptake of FITC-coupled PFCs by isolated and MACS separated EPDCs and CD11b positive cells form the infarcted heart were analyzed by determination of the mean fluorescence intensity for the gated population using flow cytometry. Briefly, suspended cells were exposed to 1% PFC emulsions in MACS buffer for 5, 10, 30 60 and 120 min in parallel at 37° C. to determine internalization and at 4° C. to measure cellular association in absence of internalization. The termination of uptake was achieved by washing with ice-cold PBS for 5 min at 500 g and analysis was performed immediately. Internalization was finally assessed by subtraction of the cellular association from the absolute data obtained from the incubation at 37° C. EPDCs isolated from hearts after MI avidly incorporated PFCs; phagocytosis reached a maximum after 50 min (see FIG. 3d). Related to a comparable cell number, CD11b⁺ cells also phagocytized PFCs as expected, however at a considerably lower rate.

1. A nanoparticle comprising one or more labeling agent(s) for use in the in vivo diagnostics of EPDCs.

2. The nanoparticle of claim 1, wherein the in vivo diagnostics is/are in vivo imaging.

3. The nanoparticle of claim 1 or 2, wherein said one or more labeling agent(s) is/are independently selected from a fluorine-containing compound, a fluorescent compound, and a genetic label.

4. The nanoparticle of claim 3, wherein said fluorine-containing compound is selected from organic and inorganic perfluorinated compounds.

5. The nanoparticle of claim 4, wherein said organic perfluorinated compound is a perfluorocarbon, particularly a perfluorocarbon selected from perfluorooctyl bromide, perfluorooctane, perfluorodecalin and perfluoro-15-crown-5-ether, particularly perfluorooctyl bromide.

6. The nanoparticle of claim 4 or 5, wherein said in vivo diagnostics are performed by means of magnetic resonance imaging, in particular 19F magnetic resonance imaging.

7. The nanoparticle of any one of claims 3 to 5, wherein said fluorine-containing compound comprises at least on 18F isotope.

8. The nanoparticle of claim 7, wherein said in vivo diagnostics are performed by PET scanning, in particular by 18F PET scanning.

9. A nanoparticle comprising one or more therapeutic agent(s) for use in the treatment of a cardiac disorder, particularly cardiac injury, cardiac ischemia or myocardial infarction.

10. The nanoparticle of claim 9, wherein said treatment comprises the differentiation of EPDCs into cardiomyocytes and/or vascular smooth muscle cells.

11. The nanoparticle of claim 10, wherein said one or more therapeutic agent(s) is/are one or more cardiomyocyte differentiation factor(s) and/or one or more vascular smooth muscle cell differentiation factor(s).

12. The nanoparticle of claim 11, wherein said one or more cardiomyocyte differentiation factor(s) and/or one or more vascular smooth muscle cell differentiation factor(s) is/are independently selected from a peptide, a protein, a nucleic acid encoding a peptide, a protein or a nucleic acid with specificity for a target nucleic acid, a nucleic acid with specificity for a target nucleic acid, and a small molecule.

13. The nanoparticle of claim 12, wherein said protein is selected from a transcription factor, a growth factor, a cytokine, a chemokine, and thymosin β4.

14. The nanoparticle of claim 12, wherein said nucleic acid encoding a peptide, a protein or nucleic acid with specificity for a target nucleic acid, is selected from a nucleic acid encoding a transcription factor, a growth factor, a cytokine, a chemokine, thymosin β4, and a miRNA.

15. The nanoparticle of claim 13 or 14, wherein said transcription factor is selected from GATA4, HAND2, MEF2C, Tbx5, Myocd, and BAF60C.

16. The nanoparticle of claim 13 or 14, wherein said growth factor is selected from transforming growth factors, particularly TGF-β and BMP.

17. The nanoparticle of claim 14, wherein said nucleic acid encoding a peptide, a protein or nucleic acid with specificity for a target nucleic acid is operatively linked with an EPDC-specific promoter, particularly an EPDC-specific promoter selected from the WT-1 promoter, the Tbx18 promoter, the Raldh-1 promoter, the Raldh-2 promoter, and the PDGF-α promoter.

18. The nanoparticle of claim 12, wherein said nucleic acid with specificity for a target nucleic acid is selected from a miRNA, and an siRNA.

19. The nanoparticle of claim 14 or 18, wherein said miRNA is selected from miRNAs 1, 132, 133, 208, 212, and 499.

20. The nanoparticle of claim 12, wherein said small molecule is selected from vitamins and ascorbic acid and retinoic acid inhibitors, particularly BMS 189453.

21. The nanoparticle according to any one of claims 1 to 20, wherein said nanoparticle has a size from about 100 nm to about 400 nm.

22. The nanoparticle according to any one of claims 1 to 21, wherein said nanoparticle is selected from a lipid-based and a polymer-based nanoparticle, in particular, said nanoparticle is selected from liposomes, polymer-drug conjugates, polymeric nanoparticles, micelles, dendrimers, polymerosomes, protein-based nanoparticles, biological nanoparticles such as viral and bacterial nanoparticles, inorganic nanoparticles and hybrid nanoparticles.

23. The nanoparticle of claim 22, wherein said nanoparticle is a unilamellar or a multilamellar liposome.

24. The nanoparticle of claim 23, wherein said one or more labeling agent(s) or said one or more therapeutic agent(s) is/are formulated as from about 0.5% to about 50%, particularly from about 1% to about 30%, more particularly form about 5% to about 20% of said labeling agent(s) or said therapeutic agent(s) emulsified in a lipid solution comprising lecithin, particularly purified egg lecithin.

25. The nanoparticle according to any one of claims 1 to 24, wherein said nanoparticle further comprises an EPDC targeting moiety.

26. The nanoparticle according to claim 25, wherein said EPDC targeting moiety is a surface structure allowing for targeting of EPDCs via epitopes of antigens, receptors or other proteins, and non-proteinaceous membrane compounds of said EPDCs.

27. The nanoparticle according to any one of claims 1 to 26, wherein said nanoparticle is for intravenous administration, injection into the pericardial sac via a catheter or injection into the injured myocardium via a catheter, particularly for intravenous administration.

28. The nanoparticle according to any one of claims 1 to 27, wherein said nanoparticle is administered after from about one to about five days after cardiac injury, particularly after from about 3 to about 4 days after cardiac injury.

29. A nanoparticle comprising one or more cardiomyocyte differentiation factor(s) and/or one or more vascular smooth muscle cell differentiation factor(s) for use as a medicament.

30. A method for analyzing EPDCs comprising the step of detecting the presence or absence of a label in EPDCs contacted with a nanoparticle according to any one of claims 1 to 8 and 21 to 28 in vitro.

31. The method according to claim 30, further comprising the step of contacting EPDCs with a nanoparticle according to any one of claims 1 to 8 and 21 to 28 in vitro.

32. A method for labeling EPDCs comprising the step of contacting EPDCs in vitro with a nanoparticle according to any one of claims 1 to 8 and 21 to 28.

33. A method for in vivo imaging of EPDCs by 19F magnetic resonance imaging or by 18F PET scanning comprising the step of administering a nanoparticle according to any one of claims 1 to 6 and 21 to 28 by intravenous injection.

34. A method for transferring one or more therapeutic agent(s) into an EPDC comprising the step of contacting said EPDC in vitro with a nanoparticle according to any one of claims 9 to 20 and 21 to 29.

35. An EPDC comprising one or more therapeutic agent(s).

36. A pharmaceutical composition comprising the EPDC cell of claim 35.

37. The EPDC of claim 35 or the pharmaceutical composition of claim 36 for use as a medicament.

38. A method for diagnosing EPDCs comprising the step of administering a nanoparticle according to any one of claims 1 to 8 and 21 to 28 to a patient.

39. A method for treating a cardiac disorder/injury comprising the step of administering a nanoparticle according to any one of claims 9 to 20 and 21 to 29 to a patient.
